(19) United States
(12) Patent Application Publication
(10) Pub. No.: US 2025/0140341 A1
(43) Pub. Date: May 1, 2025

(54) METHOD FOR HLA TYPING

(71) Applicant: NEC OncoImmunity AS, Oslo (NO)

(72) Inventors: Irantzu ANZAR, Oslo (NO); Angelina SVERCHKOVA, Oslo (NO); Richard STRATFORD, Oslo (NO); Trevor CLANCY, Oslo (NO)

(21) Appl. No.: 18/835,402

(22) PCT Filed: Feb. 4, 2022
(86) PCT No.: PCT/EP2022/052780

§ 371 Date: Aug. 2, 2024

(51) Int. Cl. G16B 20/20; G16B 30/10; G16H 20/17; G16H 20/40

(52) U.S. Cl. CPC G16B 20/20; G16B 30/10; G16H 20/17; G16H 20/40

(57) ABSTRACT

The present invention relates to a method for characterising the HLA status of a genetic sample obtained from a subject, comprising the steps of: i. carrying out DNA or RNA sequencing on said genetic sample obtained from said subject; ii. aligning the obtained sequence with one or more reference HLA allele sequences; iii. Applying a variant calling technique to identify the presence of or type of variant(s) in the HLA sequence of said genetic sample thereby to determine the HLA status.

### FIELD OF THE INVENTION

The present invention relates to methods for HLA typing, for applications in HLA matching in organ transplantation, hematopoietic stem cell blood transfusions, disease association studies, prophylactic and therapeutic vaccines in cancer and infectious diseases, and finally predicting response to immunotherapy (particularly in cancer).

### BACKGROUND TO THE INVENTION

Full-length typing of the Human Leukocyte Antigen (HLA) is a continuous challenge, as it is one of the most complex and polymorphic regions in the human genome¹⁻³. The classical HLA proteins bind in a complex with peptides that may be presented on the cell surface. Once presented at the cell surface, these complexes may then be recognized by effector T cells of the adaptive immune system. Class I HLA proteins present peptides on the surface of all human cells, and therefore identification of the precise HLA genotype has implications in organ transplantation, with crucial implications in organ transplantation, where donors and recipients need to be HLA matched⁴⁻⁶. Precise knowledge of HLA genotypes of individuals is also important in disease association studies, where HLA allelic variants have strong genetic associations to many common human diseases⁷. Additionally, variations in HLA alleles have been frequently linked to disease susceptibility in many studies^{4,8}, in addition to drug sensitivity⁹ and susceptibility to adverse drug responses¹⁰. In cancer, it has been demonstrated that specific HLA genotypes¹⁰, and in particular diversity in the HLA genotype of a patient, can predict response to immune checkpoint inhibitors (ICIs)¹¹⁻¹³.

The clinical importance of precise HLA typing is very well established, and next generation sequencing (NGS) data has recently been adopted by many diagnostic laboratories as the preferred data source to perform reliable HLA typing^{14,15}. The main outcome of HLA typing is the assignment of a unique HLA name, referred to as an HLA allele, that constitutes up to four fields of resolution separated by colons, (e.g., HLA-A*02:01:01:01). The four fields of this HLA nomenclature represent: (1) allelic group, (2) protein group, (3) synonymous DNA changes within the protein coding regions, and (4) variants in non-coding regions.

NGS-based HLA typing methods can currently be divided into two categories: HLA-targetted sequencing (e.x., PCR-based target amplification) with high sequence depth, and standard NGS (e.x., Whole Exome Sequencing (WES), Whole Genome Sequencing (WGS), and RNA-sequencing (RNA-Seq)) with moderate sequence depth. HLA-targetted sequencing (or deep sequencing) of the HLA region is the most information-dense type of NGS, and therefore most often used to resolve HLA typing ambiguities in the exons that encode the peptide binding cleft (and contribute the most information in determining the 1^{st} and 2^{nd} fields of resolution). This is due to the critical importance of determining the donor HLA-peptide complex presentation for tissue compatibility in transplantations. Although targetted HLA sequencing may be subject to PCR amplification¹⁶ in a small fraction of samples¹⁷, it is arguably considered the gold standard for HLA typing in clinical applications¹⁴. However, compared to standard NGS and especially WES based approaches; it is more laborious, expensive and time consuming¹⁴. Therefore, targetted HLA sequencing is mostly used to resolve HLA genotypes and ambiguities in the peptide binding cleft exons. Consequently most of the described HLA alleles have incomplete sequences with enriched coverage for the binding cleft exons and only a minority of the alleles come with complete and full-length HLA sequences^{18,19}.

This lack of full-length HLA sequence typing is not optimal, as identification of the complete HLA region at four fields of resolution has important medical and genomic applications. Full-length HLA sequence typing is useful, for example, to generate ancestry-based analyses²⁰ and has been shown to be critically important for identifying causal variants in HLA-based disease association studies²¹. The importance of full-length HLA sequence typing has also been shown to help optimize donor selection, improve clinical outcome, and result in fewer transplant complications, as clearly demonstrated in hematopoietic cell transplantations (HCT)²²⁻²⁶. Furthermore, full-length HLA typing may provide novel insights into the transcript expression regulation of HLA genes, including epigenetic mechanisms leading to improved understanding of complex immune diseases^{27,28}.

The advent of widely available standard NGS data has resulted in an increased number of computational NGS based HLA typing solutions^{16,19,29-32}. However, the majority of these tools perform HLA typing by identifying the closest matched HLA allele through sequence alignment of NGS reads against the references sequences in the IPD-IMGT/HLA database¹. Unfortunately, as the IPD-IMGT HLA database has sparse coverage of complete alleles typed at the 3^{rd} and 4^{th} fields of resolution, standard NGS based HLA typing solutions do not therefore reliably offer complete HLA genotypes or allow for the discovery of novel HLA alleles. Importantly, although some of these computational methods¹⁹ do provide the functionality to output full-length HLA genotypes using WES data; until quite recently³³⁻³⁵ there was credible benchmarking data available only for the protein coding sequence. De novo assembly methods do not have the limitation of relying on the IPD-IMGT HLA database, and therefore may have the potential of identifying novel HLA alleles^{32,36}. However, these tools are computationally expensive, and their accuracy is dependent on deep coverage of the HLA region and the necessity of using long reads for correct phasing.

Accordingly, there is a need for improved methods and techniques for carrying out efficient and accurate HLA typing, to characterize the HLA status of an individual, for therapeutic evaluation and intervention.

### SUMMARY OF THE INVENTION

The present invention provides an HLA typing solution that is based on alignments to known HLA sequences (e.g. at the IPD-IMGT HLA database), but simultaneously enables the discovery of novel HLA by leveraging germline and/or somatic variant calling methods. This personalized HLA typing method is applied to sequencing data and demonstrates an ability to identify novel HLA alleles and rectify HLA ambiguities, particularly at the 3^{rd} and 4^{th} fields of resolution.

According to a first aspect of the invention, there is a method for characterising the HLA status of a genetic sample obtained from a subject, comprising the steps of:
i. carrying out DNA or RNA sequencing on said genetic sample obtained from said subject;
ii. aligning the obtained sequence with one or more reference HLA allele sequences;
iii. Applying a variant calling technique to identify the presence of or type of variant(s) in the HLA sequence of said genetic sample thereby to determine the HLA status.

### DESCRIPTION OF THE DRAWINGS

The invention is described with reference to the accompanying figures, wherein:

FIG. 1 illustrates the steps taken in carrying out the invention, where NGS reads are used to perform a patient-tailored HLA typing with a HLA-centred germline calling approach.

FIG. 2 illustrates an in silico allele experiment where, after typing closest known HLA alleles, HLA-A of the individual is spiked-in using Bam Surgeon with a comprehensive set of different variant types, including single nucleotide variations, insertions and deletions.

FIG. 3 shows the visualisation of the germline variants conforming the detected novel alleles, where (a) represents single nucleotide polymorph (SNP) affecting the 3′UTR region of HLA*B allele, and (b) represents insertion affecting the first intron of HLA*DRB1 allele.

### DETAILED DESCRIPTION

The present invention discloses an HLA typing approach that relies on alignments to known HLA alleles in a reference library (e.g. the IPD-IMGT HLA database), while simultaneously also enabling the discovery of novel HLA alleles. It therefore provides a database-matching based method that is also capable of discovering novel HLA alleles. The vast majority of HLA typing tools that use the IPD-IMGT HLA database are limited to typing only known HLA alleles. Hence, the completeness of HLA typing from database-matching based methods rely on the completeness of the IPD-IMGT HLA database. This limitation is particularly problematic for HLA typing at the 3^{rd} and 4^{th} fields of resolution.

The method of the invention characterises the HLA status of a genetic sample obtained from a subject, by:
i. carrying out DNA or RNA sequencing on said genetic sample obtained from said subject;
ii. aligning the obtained sequence with one or more reference HLA allele sequences;
iii. Applying a variant calling technique to identify the presence of or type of variant(s) in the HLA sequence of said genetic sample thereby to determine the HLA status.

The term “DNA or RNA sequencing” refers to any method of sequencing DNA or RNA. DNA sequencing provides a genetic profile of the sample, while RNA sequencing reflects only the sequences that are actively expressed in the sample. DNA or RNA sequencing techniques include whole genome sequencing, exonic sequencing, Sanger sequencing and targetted HLA sequencing.

The term “exonic sequencing” refers to the sequencing of the protein-coding regions of genes in a genome. It is often referred to as “Exome sequencing” or “whole exome sequencing (WES)” and the terms can be used interchangeably herein.

Techniques for carrying out such sequencing are well known in the art. Typically, the techniques rely on the selection of only the subset of DNA that encodes proteins, referred to as exons, and the subsequent sequencing of the exonic DNA by high-throughput DNA sequencing tools.

Many different high-throughput sequencing tools exist for carrying out the exonic sequencing, and the invention is not limited by the technique used.

There are many Next Generation Sequencing platforms available from various manufacturers, including Roche 454 sequencer, Life Sciences SOLID Sequencer, Life Technologies Ion Torrent Sequencer, Illumina MiSeq, HiSeq and NovaSeq platforms, each of which can carry out massively parallel exome sequencing.

As an alternative to exonic genome sequencing “whole genome sequencing” may be used. This involves the sequencing of the whole genome of a subject, and may be carried out using the same platforms as above.

Another alternative to exonic genome sequencing is “Sanger sequencing”. The term “Sanger sequencing” and “chain termination sequencing” may be used interchangeably, and refer to the sequencing of DNA. Techniques for carrying out such sequencing are well known in the art.

Techniques typically involve the selective incorporation of chain-terminating dideoxynucleotides by a DNA polymerase during DNA replication, resulting in the generation of chain-terminated oligonucleotides due to cessation of DNA replication. Subsequent separation, for example, by gel electrophoresis, of the chain-terminated oligonucleotides enable determination of the DNA sequence.

Another alternative to exonic genome sequencing is “targetted HLA sequencing”. The term “targetted HLA sequencing” refers to the sequencing of the HLA regions of genes in a genome.

However, the preference is for the invention to be carried out with exonic sequencing.

The term “determining the HLA status” refers either to the identification of germline variants within the genetic sample obtained from a subject, wherein the subject may be a patient, or the identification of somatic variants within the genetic sample obtained a tumour from a patient.

After sequencing, the reads from the sequencing are aligned with reference HLA allele sequences. Alignment is carried out using a reference HLA allele sequence or sequences.

In the context of germline variant calling, raw sequence data are aligned to a standard reference sequence for the species of interest. Alignment to the reference sequence enables the identification of genotypes based on the assumption that most genomes are diploid.

In one embodiment, alignment may be made with respect to HLA sequences contained in accessible HLA reference libraries. In the preferred embodiment, the alignment is made with respect to the sequences contained in the databases of the International Immunogenetics Information System (IMGT; www.imt.or).

The IPD-IMGT HLA database of known HLA sequences is described in Robinson et al., Nuc Acids Res. 43, D423-31 (2015). The library may be accessed at www.ebi.ac.uk/ipd/imnt/hla with experiments disclosed herein utilising release v2.0.10.

Techniques and software for carrying out DNA or RNA sequence alignment with reference sequences are known in the bioinformatic art. Typical software programmes able to carry out alignment will be known to the skilled person. Such software includes BWA (arXiv:1303.3997[q-bio.GN]) GSNAP (Thomas et al., Bioinformatics 2010 26: 873-881) and Maq (Heng et al., Genome Research 2008 18: 1851-1858). Other suitable software packages include will be known to those of skill in the art.

In a preferred embodiment, the alignment is carried out using GSNAP.

In a preferred embodiment, the alignment with the reference HLA allele(s) is carried out using the method known as OncoHLA (Sverchkova et al. HLA 94, 504-513, 2019—reference 18) available from NEC-Oncolmmunity. The technique utilises alignment of reads against a library of reference alleles with subsequent determination of allele type based on the distribution of aligned reads and the prior probabilities of the ethnic frequencies of alleles. This incorporates GSNAP for the alignment, but also provides an estimation of allele type based on aligned reads and the prior probabilities of the ethnic frequencies of the alleles.

In the context of somatic variant calling, human reference genomes may misrepresent the true HLA alleles of individual subjects, since they feature a single sequence for each HLA gene. Consequently, in order to accurately detect somatic mutations in HLA genes from a tumour-related tissue, a reconstructed germline HLA sequence of the subject's own sample may be used as a reference sequence for alignment. For example, Germline variant calling may be carried out on normal matched tissue from the patient, such as a normal peripheral blood mononuclear cell (PBMC) to provide a reference for somatic variant calling to be carried out on the tumour-related tissue.

In one embodiment, the reference germline HLA sequence is preferably reconstructed using the method detailed in the presently-claimed invention.

In another embodiment, the reference germline HLA sequence may be reconstructed using a modified version of the method detailed in the presently-claimed invention.

After alignment is carried out the alignment reads are inputted into a tool for variant calling.

The term “germline variant calling” refers to the detection of sequence variants within the genetic sample derived from a patient, for example from a PBMC.

The term “somatic variant calling” refers to the detection of sequence variants within the genetic information of a somatic cell.

In particular, variant calling detects where the reads differ from the genome of the target region. This may be in the context of single nucleotide polymorphisms (SNPs) and short insertions and deletions (indels), as will be understood by the skilled person. In one embodiment, the method of the invention is carried out utilising one or more separate germline variant calling software tools, with only variants detected by both being selected for analysis.

In a preferred embodiment the germline variant calling is carried out to select variants that have a read depth of >10 and a variant allele frequency of >0.30. Software tools are publically available for carrying out germline variant calling, and include the Genome Analysis Toolkit GATK-Haplotypecaller (DePristo et al., Nat Genet. 2011; 43(5): 491-8), Platypus (Rimmer et al., Nat Genet. 2014; 46(8):912-8), DRAGEN (Illumina), DeepVariant (Verily Life Sciences) and Strelka2 (Kim et al., Nat Methods 2018 15: 591-594). Each may be carried out according to the developer's protocols.

GATK is a preferred method for carrying out germline variant calling as it uses state-of-the-art statistical methods (e.g. logistic regression, hidden markov model and naïve bayes classification) to identify differences between reads and the reference sequence to detect sequence differences.

Platypus is a haplotype-based method for carrying out germline variant calling as it uses local realignment of reads and local assembly to achieve high sensitivity and high specificity for efficient and accurate variant detection.

DRAGEN platform utilises highly reconfigurable field-programmable gate arrays to provide hardware-accelerated implementations of genome analysis algorithms. The algorithms are implemented as logic circuits, which provide almost instantaneous outputs to detect small variants with high analytical sensitivity and specificity.

DeepVariant is a deep learning-based method for carrying out germline variant calling that is supported in diploid organisms. The method produces pileup image tensors of aligned reads, then classifies each tensor before reporting the reads.

Strelka2 is a variant calling method that introduces a novel mixture-model-based estimation of indel error parameters from each sample, an efficient tiered haplotype-modeling strategy, and a normal sample contamination model to improve liquid tumor analysis.

In one embodiment, the method of the invention is carried out using six separate somatic variant calling software tools, with only high-quality candidates detected by at least three out of the six software tools being selected for analysis.

In a preferred embodiment the somatic variant calling is carried out to select variants that have a read depth of >10 and a minimum number of alternative (mutant) reads of 3 in the tumour data and 0 in normal data.

Software tools are publically available for carrying out somatic variant calling, and include Platypus (Rimmer et al., Nat Genet. 2014; 46(8):912-8) and Strelka2 (Kim et al., Nat Methods 2018 15: 591-594).

In addition to the germline and somatic variant calling, the method of the invention may incorporate a subsequent step of haplotype estimation (phasing). This is carried out to identify the HLA variants. Haplotypes are the two sequences of alleles that have been inherited together from an individual's parents, and resolving the genotype into the two haplotypes is desirable.

Statistical methods used for haplotype estimation are known, and computational approaches are available, including WhatsHap³⁶.

Resolution of the haplotype can then be used to determine the protein haplotype, using additional bioinformatics tools. For example, Haplosaurus³⁷ can be used to compute protein haplotype from phased variation data. Other suitable bioinformatics tools will be apparent to the skilled person.

In one embodiment, customised features may be developed to extend the functionality of the additional bioinformatics tools to annotate and fully reconstruct candidate variant HLA gene, transcript and protein sequences.

The method can be carried out to determine variation within the protein coding regions, but can also be carried out to determine variation in non-coding regions.

The sample obtained from a subject used in the methods of the invention may be any suitable biological sample. In preferred embodiments, the sample is obtained from transplant tissue (donor sample) or from blood. In some embodiments, the sample is obtained from tumour-related tissue, e.g. a cancer biopsy.

The present invention has particular utility in organ transplantation, where adaptive immunity is the main response exerted to transplanted tissue. HLA-typing is therefore an important consideration for reducing the rate of rejection or for minimising side effects. This is particularly important in bone marrow transplantation and kidney transplantation. The selection of the optimal donor is influenced by HLA typing, with the matching of donor and recipient for MHC antigens showing a positive effect on graft acceptance.

According to the present invention there is a method to determine the suitability of organ transplantation, comprising determining the HLA status of both recipient and donor by carrying out a method as described above or according to claim 1 and claims dependent thereon, and comparing the HLA status of recipient and donor to confirm a suitable HLA match.

The method is preferably carried out to identify matches in MHC Class I and MHC Class 1l, in particular, HLA-A, HLA-B, HLA-C and HLA-DR.

The method of the present invention may also be used to determine whether a subject will respond to cancer immunotherapy. In this method, the HLA status of the subject is determined by carrying out a method as disclosed above or as in claim 1 or in any claim dependent thereon, and comparing the HLA status with specific HLA genotypes known to respond to immunotherapy.

Chowell et al., Science, 2018; 359(6375):582-587, discloses that greater diversity (e.g. heterozygosity) within specific HLA molecules (e.g. class-I or HLA-I) can result in a larger repertoire of tumour-derived neoantigens to be presented to cytotoxic T-cells. Conversely, homozygosity in at least one HLA Class I locus was associated with reduced survival for checkpoint inhibitor therapy, as individual subjects homozygous in at least one HLA-I locus would be predicted to present a smaller, less diverse repertoire of tumour-derived neoantigens to cytotoxic T-cells. Somatic alterations in HLA were also shown to be indicative of a patient's response.

Accordingly, the method of the invention can be used to evaluate whether a patient undergoing, or intended to undergo, immunotherapy, such as checkpoint inhibitor therapy (e.g. with PD-1/PD-L1 or CTLA4 targetted therapeutics), will respond to the therapy. Identifying a patient homozygous in at least one HLA-I locus would be a predictor of reduced response to such therapy.

### Example

HLA Database Closest-Matched Typing from WES Data

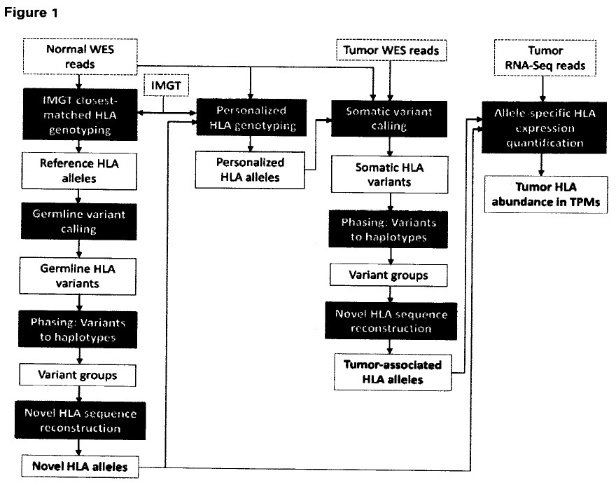

An overview of the workflow for the personalized HLA typing and tumour-specific HLA variant calling pipeline is illustrated in FIG. 1. In brief, the enormous complexity of the HLA region makes conventional mapping approaches to the reference genome result in inaccurate HLA typing. This complexity is resolved by aligning NGS reads either to a HLA reference sequence library or apply de-novo assembly methods. The method outlined in FIG. 1 is based on the former and performs HLA typing by aligning reads to the IPD-IMGT HLA database of known HLA sequences² (a method known as OncoHLA¹⁹). Once WES reads were aligned to all known HLA alleles available at the IMGT, the HLA allele was determined by the OncoHLA HLA typing tool using an integer linear programing (ILP) algorithm which uses prior probabilities of the allelic ethnic frequencies. The output of OncoHLA includes the closest-matched HLA allele from the HLA database and the associated HLA sequence, up to four fields of resolution for each allele.

Integration of Germline Variant Calling to Achieve Personalized HLA Typing from WES Data

Reads from the WES data were aligned to the reference HLA sequences outputted by OncoHLA (see FIG. 1). GSNAP³⁸ (version 2020-05-30) is used for alignment, due to its high accuracy in mapping NGS reads to highly polymorphic regions³⁹. The resulting alignment files were processed following standard practices (including sorting and duplicate marking of the reads) and provided as input into two state-of-the-art germline variant calling tools, GATK-HaplotypeCaller⁴⁰ (v4.0.6) and Strelka2⁴¹ (v2.0.10). It was reasoned in this step that each candidate germline variant is related to a potential mistyped or novel HLA allele. In order to reduce false positive calls, only variants detected by both variant callers, that also had a read depth >10 and a variant allele frequency of >0.30 were considered for further analyses.

### Integration of Ensemble Somatic Variant Calling for the Identification of Tumour Specific HLA Variants

6 different state-of-the art somatic variant calling tools were used to identify somatic mutations in the HLA region, by aligning matched tumour-normal WES reads in the HLA region against the previous typed personalized HLA sequences (known or potentially novel) (see FIG. 1). Due to the high complexity of detecting somatic variants in HLA alleles, only high-quality candidate variants reported by at least 3 out of 6 variant calling tools were considered⁴¹⁻⁴⁶. Each tool has its own algorithm and intrinsic set of rules to distinguish a variant from background noise and therefore, combining them reduces the false positive detection rate. Some additional filters were also applied to discard potential false positive calls, including minimum read depth of 10 for both tumour and normal data at a variant position, and a minimum number of alternative (mutant) reads of three in the tumour and zero in the normal data. The Ensembl Variant Effect Predictor (VEP)⁴⁷ toolkit was then used to evaluate the impact of the detected variants on the resulting gene products.

Reconstruction of Personalized HLA Alleles and Tumor-Specific HLA Alleles with the Correct Phased Haplotypes

Identifying the correct genomic phase (or haplotype) of the HLA variants was carried out for the subsequent accurate reconstruction of fully phased candidate variant HLA sequences. WhatsHap³⁶ (v0.17) was used to determine the phase relationship between heterozygous variants along two target HLA alleles. Once the phasing was conducted, Haplosaurus³⁷, a method embedded into the Ensembl Variant Effect Predictor (VEP)⁴⁷ (v95), was then used to evaluate the functional impact of the detected variants in the HLA allele sequences. Customized features were developed that extend the Haplosaurus functionality to annotate and fully reconstruct candidate variant HLA gene, transcript and protein sequences. Once the variant HLA sequences were successfully reconstructed, a compulsory additional round of HLA typing was conducted, providing the reconstructed variant HLA alleles in addition to those available in the IPD-IMGT HLA database (see FIG. 1). In this round of HLA typing, NGS reads were aligned not only to the IPD-IMGT HLA database, but also to the candidate personalized HLA variant sequences, and the closest-matching allele was assigned accordingly.

The reconstruction of tumour-specific HLA allele sequences was performed in a similar manner as that guided by germline variants, described above. However, in contrast to the germline workflow, all the generated tumour-specific HLA alleles were retained as valid potentials, since tumours may violate the diploid background assumption (due to somatic copy number alterations (CNAs) affecting the ploidy of HLA genes, or tumour heterogeneity) (see FIG. 1).

### Allele-Specific Expression Quantification

An optional step, which had particular importance to deconvolute ambiguous Class II HLA alleles generated due to suboptimal phasing of the heterozygous variants; was to perform an allele-specific expression quantification from RNA-Seq, when available. The resolution of phase using short-read NGS data was dramatically affected by the long intronic and homopolymeric regions frequent in Class II alleles (as read pairs no longer sufficiently spanned this distance). The precise mapping of RNA-Seq reads to the specific HLA sequences from the donor was relied on in order to obtain reliable expression levels for the donor's HLA alleles. For that purpose, Kallisto⁴⁸ (v0.43.1) was used for transcript isoform-level expression quantification. However, Kallisto's functionality was extended whereby previously inferred HLA genotypes were used as an index to assign RNA-Seq reads back to their corresponding HLA sequences, and then attempted to deconvolute the correct allele from the expression of the corresponding isoforms of the inferred HLA alleles. This step therefore served two purposes, first to attempt to ratify the correct HLA when phasing is not complete, and second to provide HLA allelic abundance measurements, reported as transcripts per million mapped reads (TPM).

### In Silico Spike-In of Germline Variants to Simulate Novel HLA Allele Discovery

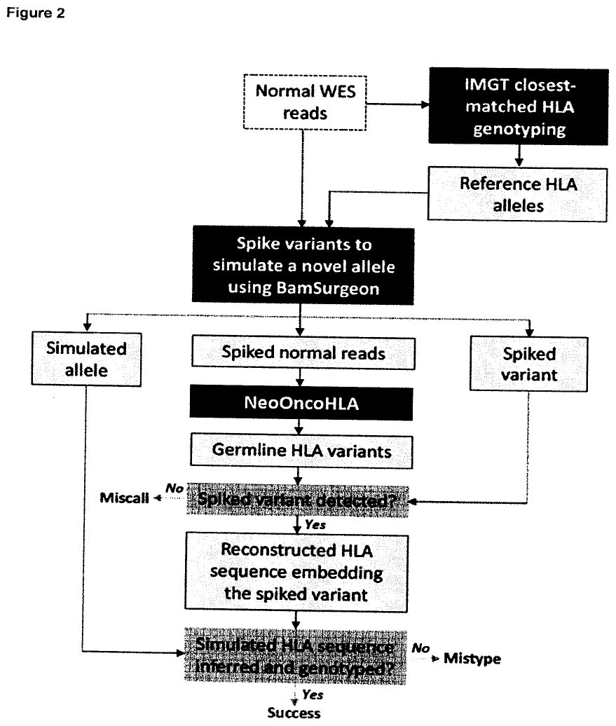

The ability of the personalized HLA typing step to infer novel or uncharacterised HLA genotypes was first evaluated on simulated novel HLA alleles (summarised here and in FIG. 2). To preserve the sequencing error profiles and complexity of biological data, and thus, keep the simulation as faithful as possible to reality, BamSurgeon⁴⁹ was used to spike germline variations into the WES data. Three different class I alleles belonging to three different normal WES patients were selected randomly to apply the simulations (HLA-A*68:01:02:01, HLA-B*51:01:01:01 and HLA-C*03:04:01:01). The WES with spiked-in variant reads were then used as input to evaluate the capability our approach to predict novel HLA alleles. In total 1800 independent simulation experiments containing 3200 variants overall were carried out including, SNPs, insertions, and deletions. A wide range of effects in the resultant protein were simulated, including missense, synonymous, in-frame, frameshift, stop gain and stop loss variants. The variants were spiked in both individually and in phased co-occurrences for the purpose of modelling more dissimilar alleles. The simulation framework first verified whether the spiked in variants were detected by the germline variant callers, if not, the experiment was labeled as a miscall. If the variant was called correctly, the variant HLA allele sequence was required to be correctly reconstructed and chosen as the best-matching HLA allele over its reference HLA counterpart. If the reference allele was outputted in this process, the experiment was labelled as an HLA mistype. These simulations were performed for a further 36 HLA-A, -B and -C alleles using 8450 spiked-in single variants to demonstrate the robustness of novel HLA allele discovery across a broader spectrum of HLA alleles.

### Simulation of Somatic Variants in HLA Allele Sequences

Following a similar workflow as in the novel HLA allele simulation experiments, the performance detecting somatic variants on HLA alleles was tested on simulated somatic variants on real data. In this case, the somatic variant was spiked in the tumour WES reads, while the normal WES reads were kept unaltered. In total, 740 simulation experiments were performed, covering the same three HLA alleles as before. The simulations included SNVs, and small insertion and deletions with a variant allele frequency (VAF) ranging from 0.01 to 0.5, allowing for the simulation of heterogeneous tumour subclones or sample contamination. The simulation results were benchmarked against POLYSOLVER (v4)⁵⁰, a state-of-the art tumour-specific HLA profiling tool. The performance of each tool was then assessed by its ability to detect each simulated somatic variant.

### Standard WES and HLA Targetted Sequencing on 10 Donor Normal PBMCs and WES on Matched Metastatic Melanoma Patients

To assess the performance of the proposed solution on clinical samples, the HLA typing pipeline was applied on 10 WES samples from the peripheral blood mononuclear cell (PBMC) of 10 metastatic melanoma donors. Additionally, WES was performed on 14 metastatic melanoma samples (matched to the 10 normal PBMCs from the same metastatic melanoma cohort⁵¹). All the research and ethics approval and permits together with the written informed consents from all the participants were obtained prior to sample collection. Exome enrichment of the samples was performed using the Agilent AllExome v5 kit, according to the vendor's protocol, and the sequencing was carried out by the Illumina HiSeq4000 system using paired-end mode with 151 bp per read and producing 50 million reads per sample, on average.

To validate the accuracy of the results, 5 samples that had at least one potentially novel class I allele were subject to targetted HLA sequencing using NGSgo-MX11-3 HLA-targetted amplification kit and analyzed with NGSengine (v2.20) (GenDx, Utrecht, Netherlands). The NGSgo-MX11-3 kit comprises amplification primers for eleven loci (including HLA-A, -B, -C, -DRB1, -DQB1, -DPB1, DRB3/4/5, DQA1, and DPA), multiplexed in three tubes, resulting in HLA locus-specific amplicons that were then used for HLA typing. In order to eliminate the possibility of analyses performed on different versions of the IPD-IPD-IMGT HLA database, both the WES and the HLA targetted sequencing analyses was performed using identical versions of the IPD-IMGT/HLA database (v.3.41.2).

### Orthogonal Validation of Somatic HLA Variants Using RNA-Seq

Validation of somatic HLA variants using WES data from 10 metastatic melanoma patients was then attempted, by also conducting an orthogonal validation using the available RNA-Seq data for the same tumour samples. Due to the nature of RNA-Seq data, only those variants located on exons regions could be subject of this validation. A variant discovered in the WES data was considered confirmed when at least one read harboring the alternate allele was found in the RNA-Seq data. A variant remained unconfirmed because of a false positive WES call, low quality of RNA-Seq data or expression down-regulation, where the allele's expression was switched off or lowly expressed. Confirming the presence of the detected variant at RNA level (when feasible), significantly reduced the probability of the variant being called erroneously due to a sequencing error.

### Results

### Simulating the Discovery of Mistyped or Novel HLA Alleles

The capability of the personalized HLA typing approach was first evaluated to capture potential mistyped or novel HLA alleles from WES data on simulated HLA variants. The HLA alleles HLA-A*68:01:02:01, HLA-B*51:01:01:01 and HLA-C*03:04:01:01 were used to perform 1800 independent simulations to assess the ability of the approach to detect mistyped or potentially novel alleles (see FIG. 2). A total of 3200 HLA variants randomly distributed across the length of the selected allele were used in the simulation. Table 1 summarizes the results, where experiments are classified according to the mutated allele, variant type and number of co-occurring simulated variants. Simulated variants were detected and the novel HLA allele was correctly inferred at a success rate of 83% across all experiments. Considering only those experiments in which single mutations were spiked in into the alleles, success rates of 97%, 93% and 96% were observed for SNVs, deletions and insertions respectively. As expected, a reduction in the success rate was observed when simulating co-occurring phased variants in the same allele. This reduction was particularly notable when simulating co-occurring indels, due to the challenge of mapping reads from multiple indel related mismatches. With respect to specific HLA genes, an 86%, 86% and 74% success rate was observed for A, B and C alleles, respectively.

TABLE 1
Summary of simulated HLA variant detection

| | Simulated | Total | | | | |
|---|---|---|---|---|---|---|
| | variant | number of | Successful | Success | | |
| Allele | combination | simulations | experiments | rate (%) | Miscall | Mistyped |
| A | SNV | 100 | 100 | 100.00 | 0 | 0 |
| | SNV x2 | 100 | 79 | 79.00 | 7 | 14 |
| | SNV x3 | 100 | 76 | 76.00 | 18 | 6 |
| | Deletion | 100 | 98 | 98.00 | 0 | 2 |
| | Deletion x2 | 100 | 62 | 62.00 | 37 | 1 |
| | Insertion | 100 | 100 | 100.00 | 0 | 0 |
| | Insertion x2 | 100 | 62 | 62.00 | 37 | 1 |
| | SNV + deletion | 100 | 99 | 99.00 | 1 | 0 |
| | SNV + insertion | 100 | 100 | 100.00 | 0 | 0 |
| B | SNV | 50 | 47 | 94.00 | 0 | 3 |
| | SNV x2 | 50 | 46 | 92.00 | 1 | 3 |
| | SNV x3 | 50 | 36 | 72.00 | 14 | 0 |
| | Deletion | 50 | 49 | 98.00 | 0 | 1 |
| | Deletion x2 | 50 | 28 | 56.00 | 22 | 0 |
| | Insertion | 50 | 50 | 100.00 | 0 | 0 |
| | Insertion x2 | 50 | 31 | 62.00 | 17 | 2 |
| | SNV + deletion | 50 | 49 | 98.00 | 0 | 1 |
| | SNV + insertion | 50 | 50 | 100.00 | 0 | 0 |
| C | SNV | 50 | 46 | 92.00 | 0 | 4 |
| | SNV x2 | 50 | 40 | 80.00 | 2 | 8 |
| | SNV x3 | 50 | 29 | 58.00 | 10 | 11 |
| | Deletion | 50 | 39 | 78.00 | 0 | 11 |
| | Deletion x2 | 50 | 23 | 46.00 | 25 | 2 |
| | Insertion | 50 | 42 | 84.00 | 0 | 8 |
| | Insertion x2 | 50 | 29 | 58.00 | 5 | 16 |
| | SNV + deletion | 50 | 41 | 82.00 | 0 | 9 |
| | SNV + insertion | 50 | 43 | 86.00 | 1 | 6 |

To demonstrate the approach's ability to identify novel alleles across a wider spectrum of HLA alleles, we extended these experiments with 8450 spiked-in single variants further simulations across a diverse range of an additional 36 HLA-A, -B and -C alleles to achieve an overall success rate of 97% (see Table 2).

TABLE 2
Extended evaluation of novel allele simulations by spiked
germline HLA variants using a broad range of HLA alleles.

| | Simulated | Total | | | | |
|---|---|---|---|---|---|---|
| | variant | number of | Successful | Success | | |
| Allele | combination | simulations | experiments | rate (%) | Miscall | Mistyped |
| A | SNV | 1108 | 1050 | 94.76 | 32 | 26 |
| | Deletion | 1195 | 1173 | 98.16 | 2 | 20 |
| | Insertion | 1099 | 1071 | 97.45 | 28 | 0 |
| B | SNV | 562 | 541 | 96.26 | 9 | 12 |
| | Deletion | 648 | 627 | 96.76 | 2 | 19 |
| | Insertion | 565 | 556 | 98.40 | 5 | 4 |
| C | SNV | 1076 | 1049 | 97.49 | 13 | 14 |
| | Deletion | 1146 | 1122 | 97.90 | 1 | 23 |
| | Insertion | 1051 | 1020 | 97.05 | 26 | 5 |

Overall, the simulations summarized in Table 1 and Table 2 indicate the potential of the proposed approach to recover the correct HLA genotypes when mistyping occurs from WES based NGS data, and thereby also the potential of this approach to identify novel HLA alleles from WES-based NGS data.

### Validation of WES Based HLA Typing at the Protein Coding Sequence Level

The OncoHLA HLA typing algorithm was then run on WES data from the PBMCs of 10 donors. For validation purposes, 5 blood samples from the 10 donors, where at least one germline variation in an HLA class I allele was predicted, were also subject to targetted HLA sequencing from the GenDx NGSgo-MX11-3 kit (see methods). The outcome of this high-resolution targetted HLA sequencing validation is described in Table 3.

TABLE 3
Overlap percentage on the validation of WES based
HLA typing versus targetted HLA sequencing

| | OncoHLA with | OncoHLA with | NeoOncoHLA with |
|---|---|---|---|
| Resolution | WES data | targetted NGS data | WES data |
| 1 field | 100 | 100 | 100 |
| 2 field | 100 | 100 | 100 |
| 3 field | 86.67 | 93.33 | 96.67 |
| 4 field | 70 | 86.67 | 86.67 |

There was a 100% overlap in the HLA typing between NeoOncoHLA and OncoHLA using WES data and HLA targetted sequencing at the protein coding sequence level (i.e., at 1^{st} and 2^{nd} field of resolution). This 100% performance overlap with HLA targetted sequencing was a validation of both NeoOncoHLA and OncoHLA, for the 1^{st} and 2^{nd} field of resolution¹⁹.

OncoHLA, the HLA typing from WES data without variant calling integration¹⁹, had a reduced performance at the 3^{rd} and 4^{th} fields, with 86.7% and 70% for the 3^{rd} and 4^{th} fields respectively (see Table 3). This reduced performance was as expected, as for any WES-based HLA typing solution, due to the lower coverage of HLA sequences in the IPD-IMGT HLA database for all HLA exons and non-coding sequences in addition to the moderate read depth of WES compared to targetted NGS. The performance for our WES based HLA typing solutions improved significantly when using deep or targetted HLA NGS data as input (see Table 3).

Integration of Germline Variant Calling Enhances Personalized HLA Typing and Enables Novel HLA Discovery from WES Data

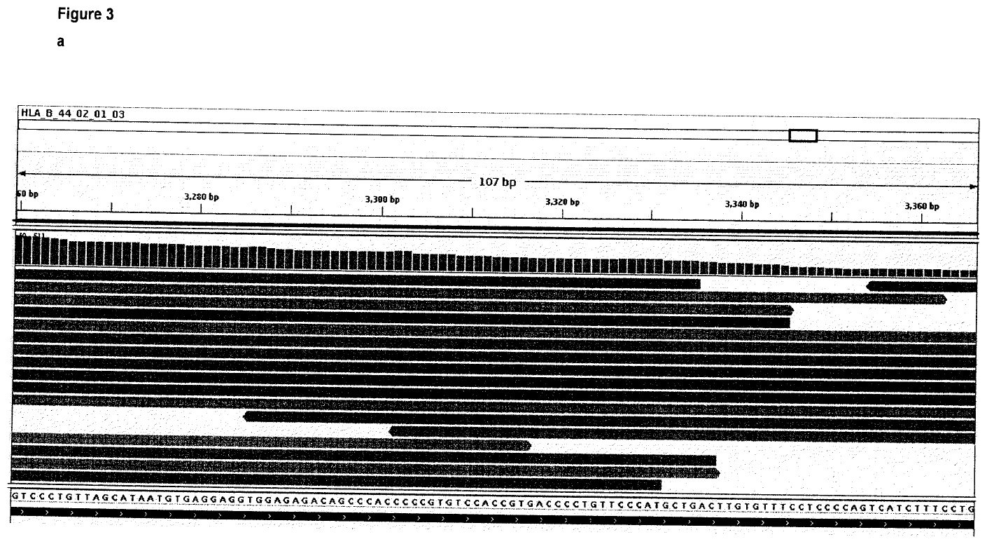

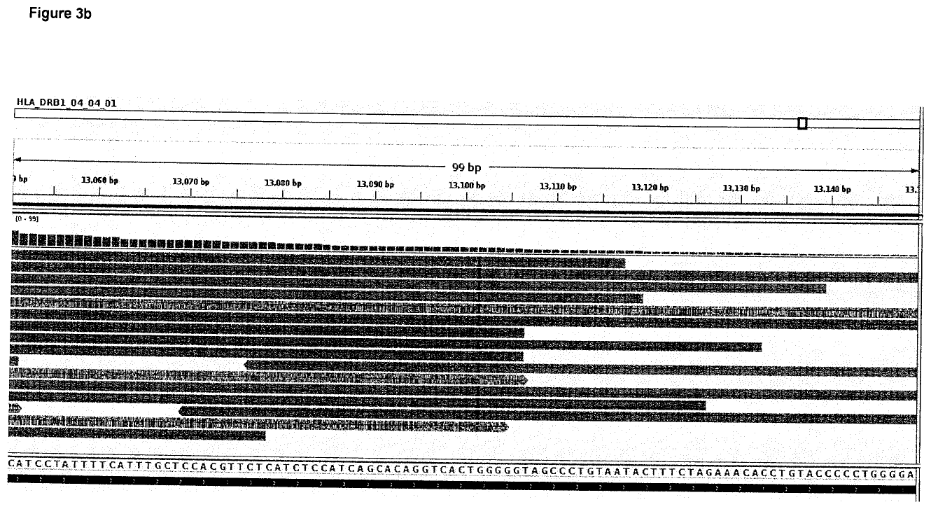

Personalized HLA typing, through the integration of variant calling (see FIG. 1), significantly improved the performance at the 3rd and 4th fields of resolution using WES data (see Table 3). It was demonstrated that the performance was enhanced through the integration of variant calling, raising the accuracy to 96.7% from 86.7%, and to 86.7% from 70% for the 3^{rd} and 4^{th} fields, respectively. In Table 4, the HLA typing results with a discrepancy using WES data versus targetted HLA sequencing data is also depicted. In all the 6 cases of variant HLA alleles among the normal PBMC samples of 5 patients, where there was at least one germline variant detected, NeoOncoHLA was able to correct the HLA mistypes. In one of these cases, the analysis led to the discovery of a novel Class I HLA allele confirmed by both WES, targetted NGS sequencing, and officially assigned the name HLA*B*:44:02:01:52 (see Table 4), and consequently improved the typing as shown in Table 3. FIG. 3 illustrates the germline variant found in the 3′UTR of the closest-matched reference HLA*B*:44:02:01:03 allele, responsible of the novel HLA*B*:44:02:01:52 allele. This Class I novel variant was also subject to confirmation at the transcriptional level from the tumour sample matched to the same patient (see Methods: “Allele-specific expression quantification”).

TABLE 4
Validating mistype correction and discovery of novel HLA
genotypes using personalized germline variant calling

| | | | Discrepancy | | Outcome of |
|---|---|---|---|---|---|
| | | | with | HLA germline | NeoOncoHLA with |
| | | GenDx HLA typing | targetted | variants | personalized |
| | OncoHLA HLA typing | using targetted HLA | HLA | detected by | germline variant |
| Sample ID | using WES data | sequencing data | sequencing | NeoOncoHLA | calling |
| UV1-0001 | HLA-B*40:01:01 | HLA-B*40:01:02:01 | 3rd field | Yes | Mistyping fixed |
| UV1-0002 | HLA-A*24:02:32 | HLA-A*24:02:01:01 | 3rd field | No | No germline |
| | | | | | variants detected |
| UV1-0002 | HLA-B*35:01:01:01 | HLA-B*35:01:01:02 | 4th field | Yes | Mistyping fixed |
| UV1-0002 | HLA-C*04:01:01:01 | HLA-C*04:01:01:13 | 4th field | No | No germline |
| | | | | | variants detected |
| UV1-0002 | HLA-C*04:01:115 | HLA-C*04:01:01:05 | 3th field | Yes | Mistyping fixed |
| UV1-0006 | HLA-B*40:01:01 | HLA-B*40:01:02:01 | 3rd field | Yes | Mistyping fixed |
| UV1-0006 | HLA-C*03:04:01:01 | HLA-C*03:04:01:02 | 4th field | No | No germline |
| | | | | | variants detected |
| UV1-0011 | HLA-B*44:02:01:03 | HLA-B*44:02:01:03 | Equal | Yes | Potential new allele |
| | | | | | characterization |
| UV1-0013 | HLA-B*52:01:01:01 | HLA-B*52:01:01:02 | 4th field | Yes | Mistyping fixed |
| UV1-0013 | HLA-C*12:02:02:02 | HLA-C*12:02:02:01 | 4th field | No | No germline |
| | | | | | variants detected |

### Somatic Variant Calling in Class I HLA Alleles Using Personalized Germline HLA Alleles as Reference

NeoOncoHLA was then applied on WES data from the 14 metastatic melanoma samples from 10 patients. To capture somatic HLA variants with improved fidelity, the variants were called by using the personalized HLA sequences derived from the matched normal PBMCs.

In total, 15 somatic mutations were detected in classical Class I alleles across the 14 melanoma samples, 7 (47%) of those were found in HLA-A, five (33%) in HLA-B and the remaining three (20%) in HLA-C alleles. The functional consequences of the somatic variants were inferred from their predicted effect on the resultant protein using VEP. In total, five out of 15 (33%) variants were situated in protein-coding regions, while 10 (66%) were in non-coding genomic regions. The predicted functional effects of the 15 detected somatic variants sorted from low to high impact were distributed as follows: five intron variants, two 5′UTR variants, three 3′UTR variants, one synonymous variant and four missense variants. One of the four missense variants occurred, interestingly, in the binding cleft of HLA-A*02:01:01:01.

The mean variant allele frequency (VAF) across the detected somatic changes was moderately low (0.13), indicating a broad genomic heterogeneity and the presence of sub-clonal mutations private to subpopulations of cancer cells. However, one of the somatic HLA variants located at HLA-A*03:01:01:01 of sample UV1-0009-T01 showed a high VAF of 0.707. This variant was annotated as missense, changing the second amino acid encoded by the allele from an alanine to valine. The variant was also confirmed in tumour RNA-Seq data, and the resultant novel somatic allele expressed with a high abundance (TPM of 914.75).

Orthogonal validation using RNA-Seq data was conducted to confirm the five somatic HLA variants on protein-coding regions (see Methods). A variant discovered in the WES data was considered confirmed when at least one read harboring the alternate allele was found in the RNA-Seq data. Three of the five exonic variants confirmed in RNA-Seq data were observed. The expression abundance of the total of four mutant HLA allele sequences was calculated using Kallisto. As expected, the mutant HLA alleles harboring those unconfirmed RNA-seq variants and with very low VAF (0.01-0.02) were not expressed. The remaining two alleles had a TPM of 7.23 and 914.76 (the latter being the variant with high VAF of 0.707 in HLA-A*03:01:01:01 of sample UV1-0009-T01, mentioned above).

POLYSOLVER applied to the same 14 metastatic melanoma samples detected 28 somatic variants compared to the 15 from NeoOncoHLA. Only one variant was called by both tools that being the high VAF (0.707) SNV, mentioned above. One source of the large difference of note was that NeoOncoHLA uses all the alleles available at IPD-IMGT/HLA database to align NGS reads, including classical Class I, classical Class II and non-classical alleles, allowing the NGS reads to map back and align to their true origin, whereas POLYSOLVER uses classical Class I alleles only. POLYSOLVER reported the same variant twice, once per typed HLA allele. This was addressed in NeoOncoHLA by being more stringent with the filters where multiple variant calling tools must detect a somatic variant compared to a single tool in POLYSOLVER, improving the mapping of the NGS reads by allowing them to map against Class II and non-classical HLA alleles, and also by including allelic variant phasing steps taking advantage of both WES and RNA-Seq data.

In the present invention, novel HLA allele discovery is made possible through the systematic integration of variant calling tools. Strong validation for HLA typing using a database-matching method is demonstrated at the 1^{st} and 2^{nd} fields of resolution, in both in silico simulations and targetted HLA sequencing experiments from the blood of the 5 donors that had at least one candidate germline variant in their HLA alleles. This validation performance was consistent with much of the recent literature on the performance of NGS based HLA typing. The reduction of the validation performance results observed at the 3^{rd} and 4^{th} fields of resolution was rectified when applying the germline variant calling strategy. This was particularly promising given that standard WES data was used to perform the HLA typing, and therefore had very sparse coverage of reads in non-coding regions (introns and UTR's), where ambiguities lie at the 4^{th} field of resolution. Interestingly, HLA non-synonymous variants were never detected in exons that encode the peptide-binding cleft in any of the analyses (i.e., in exons 2 and 3 for Class I and exon 2 for class II); reflecting the comprehensive coverage at the 1^{st} and 2^{nd} fields in the IPD-IMGT HLA database for HLA allelic variants that bestow different antigen presentation patterns. Finally, the ability of the approach described to discover new alleles was demonstrated by the detection of a novel allele not yet reported in IPD-IMGT HLA database belonging to class I (HLA-B). The novel HLA-B allele has been assigned the name B*44:02:01:52 and officially catalogued by the WHO Nomenclature Committee for Factors of the HLA System.

The approach described here is capable of effectively identifying novel Class I HLA alleles and tumour-specific HLA variants, through the systematic integration of variant calling. However, the approach is limited to alleles whose sequences share a relatively high degree of similarity to those of already known alleles in the HLA reference databases. Hence, this approach has been mostly tested on classical class I alleles, the most extensive collection of full-length allele sequences available in the IPD-IMGT HLA Database. Once more full-length HLA gene sequences become increasingly submitted, future methodologies may be carried out on class II and non-classical alleles. For the identification of highly dissimilar novel HLA alleles (as those presenting structural variants such as large insertions or deletions) from NGS data, a de novo alignment approach may be more optimal⁵². Additionally, the personalized germline HLA variant calling steps in this study were restricted to the consensus of three state-of-the-art variant callers. As numerous NGS based germline variant callers have been developed in recent years⁵³, with solutions to variant detection in complex genome regions like HLA continuously emerging^{54,55}; it may be beneficial to investigate ensemble approaches to variant calling⁵⁶, taking input from numerous germline variant callers in order to optimize the accuracy of germline variant detection in the HLA region.

A well-known source of ambiguous HLA typing results is characterized by the difficulties to always reliably reconstruct the fully phased HLA haplotypes¹⁶. This challenge can be attributed to the very nature of short-reads in WES data. To correctly phase, short NGS reads must adequately cover the variant region, which was especially difficult for Class II HLA alleles as the long homozygous stretches common in HLA alleles prevents the read pairs to reliably span the distance between the variants. Incorrect phasing is an arduous challenge in the HLA genotype field, currently, and constitutes a major source of spurious HLA typing results¹⁶. When ambiguous phasing occurred in the analyses, RNA-Seq was used to only select those reconstructed HLA alleles with RNA-Seq verification. Long reads sequencing could also be used address the challenge of haplotype phasing of HLA alleles harbouring long intronic regions.

In summary, the HLA typing approach described showed good performance for full-length HLA typing when validated using targetted HLA sequencing, and demonstrated an ability to detect novel HLA alleles. With the large amount of NGS data being continuously accumulated in many clinical projects worldwide, this approach will enable more accurate complete typing, leading to the discovery of novel HLA alleles and help fill the gaps in the IPD-IMGT/HLA database. Furthermore, using a personalized germline reference HLA genotype to perform somatic variant calling, allows tumour-specific HLA variants to be identified with increased fidelity, and help to characterize HLA associated tumour-immune escape.

### REFERENCES

1. Robinson J, Malik A, Parham P, Bodmer J G, Marsh S G: IMGT/HLA database—a sequence database for the human major histocompatibility complex. Tissue Antigens 2000, 55:280-287.
2. Robinson J, Halliwell J A, Hayhurst J D, Flicek P, Parham P, Marsh S G: The IPD and IMGT/HLA database: allele variant databases. Nucleic Acids Res 2015, 43:D423-431.
3. Horton R, Wilming L, Rand V, Lovering R C, Bruford E A, Khodiyar V K, Lush M J, Povey S, Talbot C C, Jr., Wright M W, et al: Gene map of the extended human MHC. Nat Rev Genet 2004, 5:889-899.
4. Morishima Y, Sasazuki T, Inoko H, Juji T, Akaza T, Yamamoto K, Ishikawa Y, Kato S, Sao H, Sakamaki H, et al: The clinical significance of human leukocyte antigen (HLA) allele compatibility in patients receiving a marrow transplant from serologically HLA-A, HLA-B, and HLA-DR matched unrelated donors. Blood 2002, 99:4200-4206.
5. Bray R A, Hurley C K, Kamani N R, Woolfrey A, Muller C, Spellman S, Setterholm M, Confer D L: National marrow donor program HLA matching guidelines for unrelated adult donor hematopoietic cell transplants. Biol Blood Marrow Transplant 2008, 14:45-53.
6. Lee S J, Klein J, Haagenson M, Baxter-Lowe L A, Confer D L, Eapen M, Fernandez-Vina M, Flomenberg N, Horowitz M, Hurley C K, et al: High-resolution donor-recipient HLA matching contributes to the success of unrelated donor marrow transplantation. Blood 2007, 110:4576-4583.
7. Bodmer W, Bonilla C: Common and rare variants in multifactorial susceptibility to common diseases. Nat Genet 2008, 40:695-701.
8. Trowsdale J, Knight J C: Major histocompatibility complex genomics and human disease. Annu Rev Genomics Hum Genet 2013, 14:301-323.
9. Lee M T, Mahasirimongkol S, Zhang Y, Suwankesawong W, Chaikledkaew U, Pavlidis C, Patrinos G P, Chantratita W: Clinical application of pharmacogenomics: the example of HLA-based drug-induced toxicity. Public Health Genomics 2014, 17:248-255.
10. Fan W L, Shiao M S, Hui R C, Su S C, Wang C W, Chang Y C, Chung W H: HLA Association with Drug-Induced Adverse Reactions. J Immunol Res 2017, 2017:3186328.
11. Richard C, Fumet J D, Chevrier S, Derangere V, Ledys F, Lagrange A, Favier L, Coudert B, Arnould L, Truntzer C, et al: Exome Analysis Reveals Genomic Markers Associated with Better Efficacy of Nivolumab in Lung Cancer Patients. Clin Cancer Res 2019, 25:957-966.
12. Ivanova M, Shivarov V: HLA typing meets response to immune checkpoint inhibitors prediction: A story just started. Int J Immunogenet 2021, 48:193-200.
13. Chowell D, Krishna C, Pierini F, Makarov V, Rizvi N A, Kuo F, Morris L G T, Riaz N, Lenz T L, Chan T A: Evolutionary divergence of HLA class I genotype impacts efficacy of cancer immunotherapy. Nat Med 2019, 25:1715-1720.
14. Profaizer T, Pole A, Monds C, Delgado J C, Lazar-Molnar E: Clinical utility of next generation sequencing based HLA typing for disease association and pharmacogenetic testing. Hum Immunol 2020, 81:354-360.
15. Bravo-Egana V, Sanders H, Chitnis N: New challenges, new opportunities: Next generation sequencing and its place in the advancement of HLA typing. Hum Immunol 2021.
16. Klasberg S, Surendranath V, Lange V, Schofl G: Bioinformatics Strategies, Challenges, and Opportunities for Next Generation Sequencing-Based HLA Typing. Transfus Med Hemother 2019, 46:312-325.
17. Schofl G, Lang K, Quenzel P, Bohme I, Sauter J, Hofmann J A, Pingel J, Schmidt A H, Lange V: 2.7 million samples typed for HLA by next generation sequencing: lessons learned. BMC Genomics 2017, 18:161.
18. Orenbuch R, Filip I, Comito D, Shaman J, Pe'er I, Rabadan R: arcasHLA: high-resolution HLA typing from RNAseq. Bioinformatics 2020, 36:33-40.
19. Sverchkova A, Anzar I, Stratford R, Clancy T: Improved HLA typing of Class I and Class II alleles from next-generation sequencing data. HLA 2019, 94:504-513.
20. Rotimi C N, Jorde L B: Ancestry and disease in the age of genomic medicine. N Engl J Med 2010, 363:1551-1558.
21. Zhou F, Cao H, Zuo X, Zhang T, Zhang X, Liu X, Xu R, Chen G, Zhang Y, Zheng X, et al: Deep sequencing of the MHC region in the Chinese population contributes to studies of complex disease. Nat Genet 2016, 48:740-746.
22. Flomenberg N, Baxter-Lowe L A, Confer D, Fernandez-Vina M, Filipovich A, Horowitz M, Hurley C, Kollman C, Anasetti C, Noreen H, et al: Impact of HLA class I and class II high-resolution matching on outcomes of unrelated donor bone marrow transplantation: HLA-C mismatching is associated with a strong adverse effect on transplantation outcome. Blood 2004, 104:1923-1930.
23. Mayor N P, Hayhurst J D, Turner T R, Szydlo R M, Shaw B E, Bultitude W P, Sayno J R, Tavarozzi F, Latham K, Anthias C, et al: Recipients Receiving Better HLA-Matched Hematopoietic Cell Transplantation Grafts, Uncovered by a Novel HLA Typing Method, Have Superior Survival: A Retrospective Study. Biol Blood Marrow Transplant 2019, 25:443-450.
24. Vazirabad I, Chhabra S, Nytes J, Mehra V, Narra R K, Szabo A, Jerkins J H, Dhakal B, Hari P, Anderson M W: Direct HLA Genetic Comparisons Identify Highly Matched Unrelated Donor-Recipient Pairs with Improved Transplantation Outcome. Biol Blood Marrow Transplant 2019, 25:921-931.
25. Agarwal R K, Kumari A, Sedai A, Parmar L, Dhanya R, Faulkner L: The Case for High Resolution Extended 6-Loci HLA Typing for Identifying Related Donors in the Indian Subcontinent. Biol Blood Marrow Transplant 2017, 23:1592-1596.
26. Buhler S, Baldomero H, Ferrari-Lacraz S, Nunes J M, Sanchez-Mazas A, Massouridi-Levrat S, Heim D, Halter J, Nair G, Chalandon Y, et al: High-resolution HLA phased haplotype frequencies to predict the success of unrelated donor searches and clinical outcome following hematopoietic stem cell transplantation. Bone Marrow Transplant 2019, 54:1701-1709.
27. Hosomichi K, Shiina T, Tajima A, Inoue I: The impact of next-generation sequencing technologies on HLA research. J Hum Genet 2015, 60:665-673.
28. Carapito R, Radosavljevic M, Bahram S: Next-Generation Sequencing of the HLA locus: Methods and impacts on HLA typing, population genetics and disease association studies. Hum Immunol 2016, 77:1016-1023.
29. Wittig M, Schmöhl M, Koch S, Ziemann M, Görg S, Lange V, Jacob F, Forster M, Franke A: HLAssign 2.0: An advanced Graphical User Interface for the analysis of short and long read Human Leukocyte Antigen-typing data. bioRxiv 2020:2020.2005.2025.087627.
30. Nordin J, Ameur A, Lindblad-Toh K, Gyllensten U, Meadows J R S: SweHLA: the high confidence HLA typing bio-resource drawn from 1000 Swedish genomes. European Journal of Human Genetics 2020, 28:627-635.
31. Orenbuch R, Filip I, Comito D, Shaman J, Pe'er I, Rabadan R: arcasHLA: high-resolution HLA typing from RNAseq. Bioinformatics 2019, 36:33-40.
32. Lee H, Kingsford C: Kourami: graph-guided assembly for novel human leukocyte antigen allele discovery. Genome Biol 2018, 19:16.
33. Creary L E, Guerra S G, Chong W, Brown C J, Turner T R, Robinson J, Bultitude W P, Mayor N P, Marsh S G E, Saito K, et al: Next-generation HLA typing of 382 International Histocompatibility Working Group reference B-lymphoblastoid cell lines: Report from the 17th International HLA and Immunogenetics Workshop. Hum Immunol 2019, 80:449-460.
34. Osoegawa K, Vayntrub T A, Wenda S, De Santis D, Barsakis K, Ivanova M, Hsu S, Barone J, Holdsworth R, Diviney M, et al: Quality control project of NGS HLA genotyping for the 17th International HLA and Immunogenetics Workshop. Hum Immunol 2019, 80:228-236.
35. Turner T R, Hayhurst J D, Hayward D R, Bultitude W P, Barker D J, Robinson J, Madrigal J A, Mayor N P, Marsh S G E: Single molecule real-time DNA sequencing of HLA genes at ultra-high resolution from 126 International HLA and Immunogenetics Workshop cell lines. HLA 2018, 91:88-101.
36. Patterson M, Marschall T, Pisanti N, van lersel L, Stougie L, Klau G W, Schonhuth A: WhatsHap: Weighted Haplotype Assembly for Future-Generation Sequencing Reads. J Comput Biol 2015, 22:498-509.
37. Spooner W, McLaren W, Slidel T, Finch D K, Butler R, Campbell J, Eghobamien L, Rider D, Kiefer C M, Robinson M J, et al: Haplosaurus computes protein haplotypes for use in precision drug design. Nat Commun 2018, 9:4128.
38. Wu T D, Reeder J, Lawrence M, Becker G, Brauer M J: GMAP and GSNAP for Genomic Sequence Alignment: Enhancements to Speed, Accuracy, and Functionality. Methods Mol Biol 2016, 1418:283-334.
39. Tian S, Yan H, Neuhauser C, Slager S L: An analytical workflow for accurate variant discovery in highly divergent regions. BMC Genomics 2016, 17:703.
40. Poplin R, Ruano-Rubio V, DePristo M A, Fennell T J, Carneiro M O, Van der Auwera G A, Kling D E, Gauthier L D, Levy-Moonshine A, Roazen D, et al: Scaling accurate genetic variant discovery to tens of thousands of samples. bioRxiv 2017.
41. Kim S, Scheffler K, Halpern A L, Bekritsky M A, Noh E, Kallberg M, Chen X, Kim Y, Beyter D, Krusche P, Saunders C T: Strelka2: fast and accurate calling of germline and somatic variants. Nat Methods 2018, 15:591-594.
42. Cibulskis K, Lawrence M S, Carter S L, Sivachenko A, Jaffe D, Sougnez C, Gabriel S, Meyerson M, Lander E S, Getz G: Sensitive detection of somatic point mutations in impure and heterogeneous cancer samples. Nat Biotechnol 2013, 31:213-219.
43. Koboldt D C, Zhang Q, Larson D E, Shen D, McLellan M D, Lin L, Miller C A, Mardis E R, Ding L, Wilson R K: VarScan 2: somatic mutation and copy number alteration discovery in cancer by exome sequencing. Genome Res 2012, 22:568-576.
44. Lai Z, Markovets A, Ahdesmaki M, Chapman B, Hofmann O, McEwen R, Johnson J, Dougherty B, Barrett J C, Dry J R: VarDict: a novel and versatile variant caller for next-generation sequencing in cancer research. Nucleic Acids Res 2016, 44:e108.
45. Larson D E, Harris C C, Chen K, Koboldt D C, Abbott T E, Dooling D J, Ley T J, Mardis E R, Wilson R K, Ding L: SomaticSniper: identification of somatic point mutations in whole genome sequencing data. Bioinformatics 2012, 28:311-317.
46. Erik Garrison G M: Haplotype-based variant detection from short-read sequencing. arXiv preprint 2012, 1207.3907.
47. McLaren W, Gil L, Hunt S E, Riat H S, Ritchie G R, Thormann A, Flicek P, Cunningham F: The Ensembl Variant Effect Predictor. Genome Biol 2016, 17:122.
48. Bray N L, Pimentel H, Melsted P, Pachter L: Near-optimal probabilistic RNA-seq quantification. Nat Biotechnol 2016, 34:525-527.
49. Ewing A D, Houlahan K E, Hu Y, Ellrott K, Caloian C, Yamaguchi T N, Bare J C, P'ng C, Waggott D, Sabelnykova V Y, et al: Combining tumor genome simulation with crowdsourcing to benchmark somatic single-nucleotide-variant detection. Nat Methods 2015, 12:623-630.
50. Shukla S A, Rooney M S, Rajasagi M, Tiao G, Dixon P M, Lawrence M S, Stevens J, Lane W J, Dellagatta J L, Steelman S, et al: Comprehensive analysis of cancer-associated somatic mutations in class I HLA genes. Nat Biotechnol 2015, 33:1152-1158.
51. Aamdal E, Inderberg E M, Ellingsen E B, Rasch W, Brunsvig P F, Aamdal S, Heintz K M, Vodak D, Nakken S, Hovig E, et al: Combining a Universal Telomerase Based Cancer Vaccine With Ipilimumab in Patients With Metastatic Melanoma—Five-Year Follow Up of a Phase I/IIa Trial. Front Immunol 2021, 12:663865.
52. Hayashi S, Moriyama T, Yamaguchi R, Mizuno S, Komura M, Miyano S, Nakagawa H, Imoto S: ALPHLARD-NT: Bayesian Method for Human Leukocyte Antigen Typing and Mutation Calling through Simultaneous Analysis of Normal and Tumor Whole-Genome Sequence Data. J Comput Biol 2019, 26:923-937.
53. Koboldt D C: Best practices for variant calling in clinical sequencing. Genome Med 2020, 12:91.
54. Cooke D P, Wedge D C, Lunter G: A unified haplotype-based method for accurate and comprehensive variant calling. Nat Biotechnol 2021.
55. Poplin R, Chang P C, Alexander D, Schwartz S, Colthurst T, Ku A, Newburger D, Dijamco J, Nguyen N, Afshar P T, et al: A universal SNP and small-indel variant caller using deep neural networks. Nat Biotechnol 2018, 36:983-987.
56. Anzar I, Sverchkova A, Stratford R, Clancy T: NeoMutate: an ensemble machine learning framework for the prediction of somatic mutations in cancer. BMC Med Genomics 2019, 12:63.

1. A method for characterising the HLA status of a genetic sample obtained from a subject, comprising the steps of:
i. carrying out DNA or RNA sequencing on said genetic sample obtained from said subject;
ii. aligning the obtained sequence with one or more reference HLA allele sequences;
iii. Applying a variant calling technique to identify the presence of or type of variant(s) in the HLA sequence of said genetic sample thereby to determine the HLA status.

2. The method according to claim 1, wherein the sample is obtained from tumour-related tissue of the subject.

3. The method according to claim 2, wherein the reference HLA sequence is a reconstructed germline HLA sequence derived from said subject.

4. The method according to claim 1, wherein the sample is obtained from transplant tissue.

5. The method according to claim 1, wherein the sample is obtained from blood of the subject.

6. The method according to claim 1, claim 4 or claim 5, wherein the reference HLA allele sequence is from a HLA reference library.

7. The method according to claim 6, wherein the HLA reference library is the IPD-IMGT HLA database.

8. The method according to any preceding claim, wherein the sequencing technique used is whole genome sequencing.

9. The method according to claims 1 to 7, wherein the sequencing technique used is exonic sequencing.

10. The method according to claims 1 to 7, wherein the sequencing technique used is Sanger sequencing.

11. The method according to claims 1 to 7, wherein the sequencing technique used is targetted HLA sequencing.

12. The method according to any preceding claim, wherein the HLA status of Class I HLA alleles is determined.

13. The method according to claims 1 to 11, wherein the HLA status of Class II HLA alleles is determined.

14. The method according to any preceding claim, wherein step (iii) is carried out using one or more different germline variant calling techniques.

15. The method according to claim 14, wherein the germline variant calling has a read depth of >10 and a variant allele frequency of >0.30

16. The method according to any preceding claim, wherein step (iii) is carried out using one or more different somatic variant calling techniques.

17. The method according to claim 16, wherein the somatic variant calling has a read depth of >10, and a minimum number of 3 alternative reads.

18. The method according to any preceding claim, wherein determining the HLA status includes the determination of variants within a protein coding region and/or a non-coding region.

19. The method according to any preceding claim, further comprising, after the variant calling technique, a step of determining the phase relationship or haplotype of heterozygous variants along two target alleles.

20. The method according to claim 19, wherein a variant HLA gene, transcript and/or protein sequence is reconstructed.

21. The method according to any preceding claim, wherein allele-specific expression quantification from RNA sequencing is carried out.

22. A method to determine the suitability of organ transplantation, comprising determining the HLA status of both the recipient and donor by carrying out a method according to any of claims 1 to 21 and comparing the HLA status of recipient and donor to confirm suitable HLA matching.

23. A method to determine whether a subject will respond to cancer immunotherapy, comprising determining the HLA status of the subject by carrying out the method detailed in of any of claims 1 to 21, and comparing the HLA status with specific HLA types known to respond to said immunotherapy.

24. The method of claim 23, wherein the immunotherapy is selected from:
i. checkpoint inhibition therapy;
ii. adoptive cell therapy;
iii. therapeutic T-cell cancer vaccines; or a combination thereof.
